(19) United States
(12) Patent Application Publication
(10) Pub. No.: US 2022/0280734 A1
(43) Pub. Date: Sep. 8, 2022

(54) INTAKING AN ANTIVIRAL BY INHALATION OR SPRAYING FOR RESPIRATORY TRACT INFECTION

(71) Applicant: Sultan A. M. Daham, Riyadh (SA)

(72) Inventor: Sultan A. M. Daham, Riyadh (SA)

(21) Appl. No.: 17/365,075

(22) Filed: Jul. 1, 2021

(30) Foreign Application Priority Data
Mar. 7, 2021 (SA) 121420525

(51) Int. Cl. A61M 15/08; A61M 15/00; A61M 11/00; A61K 36/534; A61K 36/54; A61K 9/70

(52) U.S. Cl. CPC A61M 15/08; A61M 15/009; A61M 11/008; A61K 36/534; A61K 36/54; A61K 9/7023; A61M 2202/0007

(57) ABSTRACT

Methods of treatment to inhibit or kill microorganisms (Bacteria or Virus) from infecting a respiratory tract of a human patient and to treat the respiratory tract when already infected by the microorganism are described. The method of treatment may include the step of administering a therapeutically effective dose of a treatment medication comprising eugenol to the patient via a nasal passageway into a respiratory system of the patient. Eugenol is found in clove oil, cinnamon oil, and other essential oils. Any one or more of these eugenol-containing oils may be used.

### STATEMENT OF RELATED APPLICATIONS

This application claims priority from Saudi Arabian patent application Serial No. 121420525 filed on Mar. 7, 2021

### FIELD OF THE INVENTION

The present invention relates to the inhalation of treatment medications in patients that require either prophylactic treatment to prevent respiratory infection by viruses or curative treatment for certain respiratory infections by viruses.

### BACKGROUND

It is generally accepted in modern medicine that administration of some medications, whether natural or synthetic, are necessary from time to time to maintain good health or cure ill health. In environments where there is an increased level of air pollution (particulates and chemicals), including air-borne germs (bacteria and viruses), it has often become necessary to treat sensitive patients prophylactically, and of course to treat already-afflicted patients. In addition, it has become more commonly known recently, due to the outbreak of coronavirus strain, Covid 19, that precautions are required to avoid the transmission of disease by air-borne germs spread between people. It is known, for example, that in large close-proximity gatherings, in workplaces, supermarkets, stadiums, and public transportation, air-borne germs spread readily between persons in proximity to each other because they inevitably will inhale minute droplets emitted from another person who is infected. The use of face masks as protective equipment to prevent the wearer from inhaling these very fine air-borne droplets that ay contain a viral or bacterial load is sometimes not practical or may be socially inconvenient.

There exists a need for a practical method useful in a variety of social settings to prevent minute droplets carrying a viral or bacterial load from being inhaled by a person not wearing a mask or other protective gear. Further, the method desirably should not obstruct the nose (or mouth) to significantly impair breathing of the user and should not require constant adjustment or hand-touching by the user.

### SUMMARY

The present technology provides a wide range of benefits and advantages over the prior art. For example, as described in more detail here below, the treatment medication can be easily placed in the nasal cavity and does not obstruct breathing through the nose. The treatment does not obstruct the mouth and requires no constant adjustment or manipulation by hand once in place. The treatment medication can be used by anyone, including athletes or gym users, during sports.

The treatment medication can be used for curative treatment purposes (i.e., post-infection) and for prevention purposes (i.e. as a prophylactic measure). In addition, and without limitation, the treatment medication can be configured for use as a spray to be administered to the nasal passage, for example in a similar way as a nasal spray.

Methods of treatment include, but are not limited to, administering to a patient in need, a therapeutic dose of a treatment medication, continuously or at prescribed intervals, for inhalation via the nasal passageway such as to reach the respiratory tract where the treatment medication dwells and acts there as an anti-viral medication.

There is provided an exemplary method of treatment to inhibit microorganisms from infecting a respiratory tract of a human patient and to treat the respiratory tract when already infected by the microorganism, whereby the method of treatment includes the step of administering a therapeutically effective dose of a treatment medication comprising eugenol to the patient via a nasal passageway into a respiratory tract of the patient.

In an exemplary embodiment of the method, the treatment medication may include clove oil.

In another exemplary embodiment, the treatment medication may include cinnamon oil.

In a further exemplary embodiment, the treatment medication may include a mixture of clove oil and cinnamon oil.

Yet further, in exemplary embodiments, the treatment medication may include a combination of clove oil and cinnamon oil.

In an exemplary embodiment, the method of treatment may include administering an additive to facilitate human inhalation. The additive may include menthol.

In exemplary embodiments, the dose may be administered by spraying the treatment medication into a nasal passageway of the patient.

In another exemplary embodiment, the method may include placing a medication carrier in or near a nasal passageway of the patient to disperse the treatment medication into the nasal passageway thereby to administer the dose.

A further exemplary embodiment may include leading a tube from a container of the treatment medication into a nasal passageway of the patient such that inhalation by the patient draws a dose of treatment medication from the container into a respiratory tract of the patient.

In an exemplary embodiment, the method of administering a dose may include using a spray squeeze bottle to spray the treatment medication into a nasal passageway of the patient.

Moreover, the method may include treatment medication of clove oil in the concentration range 0.00001 to 100% v/v.

In another embodiment, the method includes administering a dose of cinnamon oil in the concentration range 0.00001 to 100% v/v.

In an exemplary embodiment, for delivery to the respiratory tract, via inhalation, the eugenol may be diluted with a suitable non-toxic and non-reactive diluent to about 20% v/v concentration.

A further embodiment provides adding the treatment medication that includes eugenol to a patch for transdermal delivery of the eugenol, in a continuous manner. In this instance additive may be added to facilitate the transdermal application of the eugenol.

Further embodiments are directed to any natural products or chemicals including eugenol used as an antiviral internally or externally. And are directed to any methods used as an antiviral (external or internal), including eugenol.

The exemplary embodiments present both methods of prophylactic treatment of a respiratory tract of a human patient, as well as methods of treatment of an infected respiratory tract of a human patient, using a treatment medication that contains eugenol, and that is administered via the nasal cavity to the respiratory tract.

### DETAILED DESCRIPTION

The present technology presents a treatment method and treatment medication that can be used either as (1) a curative treatment for persons infected in respiratory tract with a virus or bacteria, or (2) as a prophylactic treatment to prevent a bacterial or viral infection, or both.

The treatment medication that includes eugenol kills or inhibits microorganisms residing in the nasal cavity and the trachea, thereby inhibiting infection of the lungs. The delivered treatment medication, upon reaching the lungs, also facilitates killing or inhibiting microorganisms already in the lungs.

The treatment medication includes a natural oil that has eugenol as a component. For example, and without limitation, clove oil and cinnamon oil each comprise eugenol. Eugenol is an allyl chain-substituted guaiacol, a member of the allylbenzene class of chemical compounds. It is a colorless to pale yellow, aromatic oily liquid extracted from certain essential oils, especially from dove oil, cinnamon and some essential oils. Accordingly, embodiments of the treatment medication may include any one or more of these essential oils. Eugenol is present in high concentrations in clove oil. Eugenol has a pleasant, spicy, clove-like scent. The name eugenol is derived from Eugenia carophyllata, the former Linnean nomenclature term for cloves, (The currently accepted name is Syzygium aromaticum.)

According to exemplary embodiments, clove oil can be used alone as the treatment medication as antivirus. In other exemplary embodiments it may be used together with cinnamon oil as the treatment medication as antivirus. In exemplary methods of administration, the treatment medication may be administered via the nasal passageway and thereby into the respiratory tract. This mode of administration, in therapeutically effective amount, prevents respiratory tract infection (i.e., inhibits infection) through inhalation of the microorganism (herein defined to include both viruses and bacteria) that adversely affects the respiratory tract, For example, the microorganism that attacks the respiratory tract may include, but is not limited to, a virus such as a coronavirus (exemplified by colds, influenza, and covid-19 viruses) or any other virus or bacterium that primarily affects the respiratory tract.

In an exemplary embodiment, the daily dose of eugenol to be supplied to the patient for daily therapeutic effectiveness is to use a conventional nasal spray squeeze bottle and to deliver two sprays in each nostril every four hours for 24 hours only. For prevention, deliver one spray in each nostril three times a day. The dose may be diluted with any suitable diluent, to a 20% v/v concentration. The dose may be delivered continuously, or in partial doses at selected periodic intervals, for example hourly or every three hours, etc. For example, in the event of a protocol of periodic dosing, of the daily dose is delivered n times per day, where n can vary as a practical matter, from about 3 to about 24. It should be noted that clove oil and cinnamon oil are available at a range of concentrations of eugenol, which is the active ingredient. As a consequence, the therapeutic dose is based on eugenol quantity which can be calculated based on the concentration of eugenol in the clove oil or cinnamon oil, or other oils disclosed herein.

In an exemplary embodiment, administration of the treatment medication into the nasal passageway may be carried out continuously using the inhalation of the patient to draw the treatment medication from the nasal cavity through the nasal passageway and thence to the lungs. In this exemplary embodiment, the daily dose of medication is administered continuously and the rate of treatment medication entering the nasal passageway is controlled to correspond to the required dose. This controlled continuous dosage may he effectuated by control of the concentration of the treatment medication being inhaled, for example, or by flow control of the quantity of treatment medication flowing, or both. The method is advantageous and can be practiced by several techniques. For example, by use of a device like a nasal cannula that has the end of the delivery tube in an upper section of a bottle containing the treatment medication, where vapor from the treatment medication accumulates. Thus, during normal breathing, upon inhalation, vapor is drawn into the tube to the cannula and into the nasal passageway to the lungs. Of course, other techniques may also be used to provide a continuous supply of treatment m edication.

As indicated above, in some exemplary embodiments, the treatment medication may be provided by a spray bottle. Alternatively, bottles equipped with metering pumps, that control the dose, such as the dose-control led pump bottles that are commonly used for nasalallergy sprays, may also be used.

In another exemplary embodiment, diluents and additives that are not harmful or toxic to the respiratory tract may be added to the treatment medication. Additives such as (but not limited to) menthol may further aid the patient by facilitating or easing the act of breathing.

A further embodimentprovides adding the treatment medication that includes eugenol to a patch for transdermal delivery of the eugenol, in a continuous manner. In this instance additive may be added to facilitate the transdermal application of the eugenol. The eugenol may be derived from natural sources.

The foregoing description provides details of the inventive technology and presents exemplary embodiments. The description does not limit the scope of the inventive technology which is only limited by the scope of the appended patent claims as interpreted by a court of competent jurisdiction.

1. A method, comprising:
administering an antiviral treatment by inhalation to kill viruses infecting a respiratory tract of a human patient, comprising:
administering a therapeutically effective dose of a treatment medication in a nasal passageway of the human patient to or kill the viruses, the treatment medication comprising eugenol as an active ingredient such that as the human patient breathes, the eugenol is drawn via a nasal passageway into the respiratory tract of the human patient, wherein the eugenol is an active ingredient
wherein a source of the eugenol as the active ingredient is either cinnamon oil or clove oil or a mixture of the cinnamon oil and the clove oil,
and wherein the treatment medication comprises at least 20% v/v concentration.

2.-4. (canceled)

5. The method of claim 1, wherein the step of administering the treatment medication comprising the eugenol comprises administering a combination of any two or more of the clove oil, the cinnamon oil, and other essential oils.

6. (canceled)

7. The method of claim 1, wherein the step of administering the treatment medication comprises administering menthol.

8. The method of claim 1, wherein the step of administering the therapeutically effective dose includes providing the therapeutically effective dose via inhalation after spraying the treatment medication into the nasal passageway of the human patient.

9. The method of claim 1, wherein the step of administering the therapeutically effective dose comprises leading a tube from a container of the treatment medication into the nasal passageway of the human patient such that inhalation by the human patient draws the treatment medication from the tube of the container into the respiratory tract of the human patient.

10. (canceled)

11. The method of claim 1, wherein the step of administering the therapeutically effective dose includes administering the therapeutically effective dose when the human patient is already infected by a virus of the viruses.

12. The method of claim 1, wherein the step of administering the therapeutically effective dose includes administering the therapeutically effective dose continuously or at prescribed intervals for inhalation via the nasal passageway to reach the respiratory tract, wherein the treatment medication dwells in the respiratory tract and acts as an anti-viral medication.

13. A method, comprising:
administering a continuous method of treatment of a respiratory tract of a human patient to kill viruses, the continuous method of the treatment comprising:
administering a therapeutically effective dose of a treatment medication comprising eugenol to the human patient by depositing the therapeutically effective dose into a nasal passageway, such that the therapeutically effective dose is drawn into a respiratory tract of the human patient in order to kill the viruses, wherein the depositing is by using inhalation or spraying by the human patient during breathing to draw the treatment medication into the respiratory tract, wherein the eugenol is an active ingredient,
wherein a source of the eugenol as the active ingredient is either cinnamon oil or clove oil or a mixture of the cinnamon oil and the clove oil,
and wherein the treatment medication comprises at least 20% v/v concentration.

14. The method of claim 13, wherein the treatment medication dwells in the respiratory tract and acts as an anti-viral medication.

15-20. (canceled)
